(19) United States
(12) Patent Application Publication
(10) Pub. No.: US 2014/0135289 A1
(43) Pub. Date: May 15, 2014

(54) METHOD FOR PREVENTING BRAIN ATROPHY

(71) Applicant: NIPPON SUISAN KAISHA, LTD., (US)

(72) Inventors: Li HAN, Tokyo (JP); Tomoko TSUJI, Tokyo (JP); Pengtao LI, Beijing (CN); Jun WANG, Beijing (CN); Hongtao LEI, Beijing (CN)

(73) Assignee: NIPPON SUISAN KAISHA, LTD., Tokyo (JP)

(21) Appl. No.: 13/857,518

(22) Filed: Apr. 5, 2013

(30) Foreign Application Priority Data
Apr. 5, 2012 (CN) PCT/CN2012/073535

(51) Int. Cl. A61K 31/685; A61K 35/56

(52) U.S. Cl. CPC A61K 31/685; A61K 35/612
USPC 514/77

(57) ABSTRACT

The present invention provides brain atrophy prevention agent comprising a phospholipid containing a highly unsaturated fatty acid as a constituent fatty acid, as an active ingredient.

### TECHNICAL FIELD

The present invention relates to a method to prevent or improve brain atrophy that occurs in conjunction with aging or the like. The present invention also relates to a method of treating disorders related to brain atrophy such as Alzheimer's disease, or to a method for preventing these disorders.

### BACKGROUND ART

Brain atrophy occurs as a result of shrinking of brain volume by the death of neurons of the brain. Normally, brain atrophy progresses in conjunction with aging and is associated with symptoms such as forgetfulness, and is confirmed by measuring the brain volume using magnetic resonance imaging (MRI) or the like. Research results have been reported both in and outside of Japan showing that the intake of large quantities of alcohol promotes brain atrophy.

Dementia which causes reduction in brain function due to acquired brain organic disorder such as brain atrophy is a disease with symptoms such as memory impairment and cognitive dysfunction and the like. Of all dementia, Alzheimer's type dementia (Alzheimer's disease) is a progressive neurodegenerative disease with major symptoms such as progressive memory impairment and cognitive dysfunction, and the morbidity rate has continued to accelerate in recent years.

Alzheimer's disease is known to have symptoms that progress in conjunction with the occurrence and severity of diffuse brain atrophy. When diagnosing Alzheimer's disease, characteristic atrophy of the cerebrum such as enlarged brain fissures and enlarged lateral ventricle has been observed in imaging tests such as CT and MRI and the like.

Therapeutic agents for Alzheimer's disease such as cholinesterase inhibitors have been developed in recent years, and pharmaceutical treatments are widely used (Patent Documents 1 through 4).

### CITATION LIST

### Patent Documents

Patent Document 1: WO2007/091613

Patent Document 2: Japanese Unexamined Patent Application Publication (Translation of PCT Application) No. 2010-501566

Patent Document 3: Japanese Unexamined Patent Application Publication (Translation of PCT Application) No. 2009-501224

Patent Document 4: Japanese Unexamined Patent Application Publication No. 2005-263734

### SUMMARY OF INVENTION

### Technical Problem

Therapeutic agents for Alzheimer's disease that accompanies brain atrophy such as cholinesterase inhibitors are widely used, but these cannot be considered fundamental therapeutic agents for brain atrophy, and at the current time, a problem remains in that a sufficient treatment method for brain atrophy has not been established.

An object of the present invention is to provide means for preventing brain atrophy associated with aging or the like and brain atrophy that occurs in neurological disorders such as Alzheimer's disease and the like. Furthermore, an object of the present invention is to provide a brain atrophy prevention agent that is safer and can be used as a food, beverage, or supplement.

### Solution to Problem

As a result of diligent research performed in order to achieve the aforementioned objects, the present inventors have discovered that phospholipids have a brain atrophy preventing effect and completed the present invention.

The present invention provides the following brain atrophy prevention agents (1) to (15).

(1) A brain atrophy prevention agent, comprising, as an active ingredient, a phospholipid containing a highly unsaturated fatty acid as a constituent fatty acid.

(2) The brain atrophy prevention agent described in (1), wherein the highly unsaturated fatty acid is an n-3 highly unsaturated fatty acid.

(3) The brain atrophy prevention agent described in (2), wherein the n-3 highly unsaturated fatty acid is eicosapentaenoic acid or docosahexaenoic acid.

(4) The brain atrophy prevention agent described in any of (1) to (3), wherein the phospholipid is selected from the group consisting of phosphatidylcholine, phosphatidylserine, phosphatidylethanolamine, phosphatidic acid, phosphatidylglycerol, and phosphatidylinositol.

(5) The brain atrophy prevention agent described in (4), wherein the phospholipid is a phosphatidylserine.

(6) The brain atrophy prevention agent described in any of (1) to (5), comprising, as an active ingredient, a refined krill oil and/or a product of enzyme reaction with a krill oil as a substrate.

(7) A brain atrophy prevention agent comprising, as an active ingredient, a refined krill oil and/or a product of enzyme reaction with a krill oil as a substrate.

(8) The brain atrophy prevention agent described in (7), wherein the refined krill oil and/or the product of enzyme reaction with a krill oil as a substrate comprises a phospholipid that contains a highly unsaturated fatty acid as a constituent fatty acid.

(9) The brain atrophy prevention agent described in (8), wherein the highly unsaturated fatty acid is an n-3 highly unsaturated fatty acid.

(10) The brain atrophy prevention agent described in (9), wherein the n-3 highly unsaturated fatty acid is eicosapentaenoic acid or docosahexaenoic acid.

(11) The brain atrophy prevention agent described in any of (8) to (10), wherein the phospholipid is selected from the group consisting of phosphatidylcholine, phosphatidylserine, phosphatidylethanolamine, phosphatidic acid, phosphatidylglycerol, and phosphatidylinositol.

(12) The brain atrophy prevention agent described in (11), wherein the phospholipid is a phosphatidylserine.

(13) The brain atrophy prevention agent described in any of (1) to (12) for administering the lipid to a subject at a dosage of 1 to 10,000 mg/50 kg body weight/day, preferably 1 to 5,000 mg/50 kg body weight/day.

(14) The brain atrophy prevention agent described in any of (1) to (13) for use in preventing brain atrophy due to aging.

(15) The brain atrophy prevention agent described in any of (1) to (13) for use in treating or preventing Alzheimer's disease.

The following methods (16) to (19) are provided according to another aspect of the present invention.

(16) A method for preventing brain atrophy, which comprises oral administration to animals of the brain atrophy prevention agent described in any of (1) to (15).

(17) A method for treating or preventing Alzheimer's disease, which comprises oral administration to animals of the brain atrophy prevention agent described in any of (1) to (15).

(18) A method for improving brain atrophy, which comprises oral administration to animals of the brain atrophy prevention agent described in any of (1) to (15).

(19) A method for recovering brain atrophy, which comprises oral administration to animals of the brain atrophy prevention agent described in any of (1) to (15).

The following uses (20) to (23) are provided according to another aspect of the present invention.

(20) Use of a phospholipid containing a highly unsaturated fatty acid as a constituent fatty acid in preparation of a medicament for preventing brain atrophy.

(21) Use of a phospholipid containing a highly unsaturated fatty acid as a constituent fatty acid in preparation of a medicament for treating or preventing Alzheimer's disease.

(22) Use of a refined krill oil and/or a product of enzyme reaction with a krill oil as a substrate in preparation of a medicament for preventing brain atrophy.

(23) Use of a refined krill oil and/or a product of enzyme reaction with a krill oil as a substrate in preparation of a medicament for treating or preventing Alzheimer's disease.

The following food, animal feed, or pharmaceutical preparation of (24) is provided according to another aspect of the present invention.

(24) A food, an animal feed, or a medicament comprising the brain atrophy prevention agent described in any of (1) to (15).

The following uses of (25) to (36) are provided according to another aspect of the present invention.

(25) A method for preventing brain atrophy, comprising administrating to a subject an effective amount of a phospholipid containing a highly unsaturated fatty acid as a constituent fatty acid,.

(26) The method according to (25), wherein the highly unsaturated fatty acid is an n-3 highly unsaturated fatty acid.

(27) The method according to (26), wherein the n-3 highly unsaturated fatty acid is eicosapentaenoic acid or docosahexaenoic acid.

(28) The method according to (25), wherein the phospholipid is selected from the group consisting of phosphatidylcholine, phosphatidylserine, phosphatidylethanolamine, phosphatidic acid, phosphatidylglycerol, and phosphatidylinositol.

(29) The method according to (28), wherein the phospholipid is a phosphatidylserine.

(30) The method according to (25), wherein the phospholipid is a refined krill oil or a product of enzyme reaction with a krill oil as a substrate.

(31) The method according to (30), wherein the refined krill oil contains 40 wt % or more of phospholipids, and a ratio of n-3 highly unsaturated fatty acid in the total fatty acid is 20 wt % or more.

(32) The method according to (30), wherein the refined krill oil contains 90 wt % or more of diacylglycerophospholipids and 6 wt % or less of lysoacylglycerophospholipids in the phospholipid composition thereof. (33) The method according to (30), wherein the refined krill oil or the product of enzyme reaction with a krill oil as a substrate is:

a krill oil that is obtainable by a process comprising: obtaining a squeezed liquid by squeezing a whole krill or a part thereof, heating the squeezed liquid to a temperature at which proteins contained in the squeezed liquid coagulate, carrying out solid-liquid separation so as to separate the heated squeezed liquid into a solid component that contains lipid components and an aqueous component that contains water-soluble components, washing the resulting solid containing lipids or a dried product thereof with water, dehydrating and/or drying, and then extracting lipids from the solid containing lipids or the dried product thereof; or

a phosphatidylserine-containing oil/fat produced with the krill oil as a raw material.

(34) The method according to (25), wherein the dosage of the phospholipid is from 1 to 10,000 mg/50 kg body weight/day.

(35) The method according to (25), wherein the brain atrophy occurs in conjunction with aging.

(36) The method according to (25), wherein the brain atrophy occurs in conjunction with Alzheimer's disease.

### Advantageous Effects of Invention

The brain atrophy prevention agent of the present invention has a significant preventing effect for brain atrophy due to aging and for brain atrophy that is characteristically observed with Alzheimer's disease and the like. Furthermore, the brain atrophy prevention agent of the present invention contains naturally derived ingredients as active ingredients, and therefore has high safety and is suitable for long-term administration.

### BRIEF DESCRIPTION OF DRAWINGS

FIG. 1 is a diagram illustrating a test process for test example 1.

FIG. 2 is a photograph of a device that is used for step down test in test example 1.

FIG. 3 is a graph illustrating the number of errors for each group during step down test in test example 1.

FIG. 4 is a graph illustrating the error time for each group during step down test in test example 1.

FIG. 5 is a photograph of a device that is used for Y maze test in test example 1.

FIG. 6 is a graph illustrating the test results for each group during Y maze test in test example 1.

FIG. 7 is a graph illustrating AMP10 animal half brain weight for 45 day Cont. groups and 90 day Cont. groups in test example 1.

FIG. 8 is a graph illustrating the half brain front part weight for each group in test example 1.

FIG. 9 is a graph illustrating the whole brain weight ratio for each group in test example 1.

FIG. 10 is a graph illustrating the Nissl body density of the hippocampus of each group in test example 1.

FIG. 11 is a graph illustrating the Nissl body density of the cerebral cortex of each group in test example 1.

FIG. 12 is a graph illustrating the concentration of IGF-1 in the brain for each group after 45 days in test example 1.

FIG. 13 is a graph illustrating the concentration of SOD in the brain for each group after 45 days in test example 1.

FIG. 14 is a graph illustrating the concentration of MDA in the brain for each group after 45 days in test example 1.

FIG. 15 is a diagram illustrating a test process for test example 2.

FIG. 16 is a graph illustrating the number of errors for each group during step down test in test example 2.

FIG. 17 is a graph illustrating the latency time for each group during step down test in test example 2.

FIG. 18 is a schematic illustration for showing the proximity of point C as a brain tissue fragment in the removed whole brain in test example 2.

FIG. 19 is a graph illustrating the cross-sectional area of the neocortex of each group in test example 2.

FIG. 20 is a graph illustrating the thickness of the neocortex of each group in test example 2.

FIG. 21 is a graph illustrating the Nissl body density of each group in test example 2.

FIG. 22 is a graph illustrating the concentration of Iba-1 in the brain for each group in test example 2.

FIG. 23 is a graph illustrating the concentration of IGF-1 in the brain for each group in test example 2.

FIG. 24 is a graph illustrating the concentration of GSH-Px in the brain for each group in test example 2.

FIG. 25 is a graph illustrating the concentration of MDA in the brain for each group in test example 2.

FIG. 26 indicates the results of HPLC analysis of the starting material and product of the reaction described in Example 3.

FIG. 27 indicates the results of HPLC analysis of krill oils obtainable by the procedure described in Example 1 and those commercially available.

### DESCRIPTION OF EMBODIMENTS

Hereinafter, the present invention is described in more detail.

The present invention provides brain atrophy prevention agent including a phospholipid including a highly unsaturated fatty acid as a constituent fatty acid as an active ingredient. The brain atrophy prevention agent of the present invention may include a component of krill origin including a phospholipid such as, for example, a ground product of a krill, a krill meal, krill meat, or the like.

The brain atrophy prevention agent of the present invention contains an effective amount of phospholipids. Herein, the term “effective amount” refers to the amount required in order to demonstrate the effect of preventing brain atrophy. For example, from 1 to 5,000 mg/1 kg of body weight, preferably from 2.5 to 2,500 mg/1 kg of body weight, and particularly preferably from 10 to 1,000 mg/1 kg of body weight of an animal per day. Especially in cases of human adults, the effective amount is from 1 to 10,000 mg/50 kg of body weight, preferably from 2.5 to 5,000 mg/50 kg of body weight, more preferably from 5 to 3,000 mg/50 kg of body weight, and particularly preferably from 10 to 1,000 mg/50 kg of body weight per day. In cases of human adults, it is preferable that a greater amount of the lipid be ingested to achieve more prominent brain atrophy preventing effects, but if too great, undesirable characteristics such as becoming excessively oily, absorption lagging, dyspepsia, indigestion, loss of appetite, and the like will occur. These ingestion amounts may be an amount ingested at one time or may be an amount ingested multiple times, for example, two or three times.

“Phospholipid” refers to a substance in which at least one of the three hydroxyl groups of the glycerol is ester bonded with the fatty acid and the other one hydroxyl group is covalently bonded with a phosphate. The phosphates ordinarily covalently bond with the first or the third hydroxyl group of the glycerol. Amounts of the triacylglycerol and the phospholipid as the biological lipid are great and are important.

Phospholipids are known as major components constituting cell membranes and have a hydrophilic phosphate part and a hydrophobic fatty acid part. Phospholipids are divided into diacylglycerophospholipids having the fatty acid parts at a first position and second position of the glycerol backbone and lysoacylglycerophospholipids. Lysoacylglycerophospholipids are divided into 1-acylglycerol lysophospholipids having the fatty acid part only at the first position on the glycerol backbone, and 2-acylglycerol lysophospholipids having the fatty acid part only at the second position of the glycerol backbone. In the present specification, “phospholipid” includes all of these, but the diacylglycerophospholipid is particularly preferable. Examples of the diacylglycerophospholipid include phosphatidylcholine (PC), phosphatidylethanolamine (PE), phosphatidylserine (PS), phosphatidylinositol (PI), phosphatidylglycerol (PG), cardiolipin (CL), phosphatidic acid (PA), and mixtures of two or more thereof; preferably PC, PE, PS, PI, PA, and mixtures of two or more of thereof; and particularly preferably PC, PS, or a mixture thereof. Examples of the lysoacylglycerophospholipid include 1- or 2-lyso PC, 1- or 2-lyso PE, 1- or 2-lyso PS, 1- or 2-lyso PI, 1- or 2-lyso PG, 1- or 2-lyso CL, 1- or 2-lyso PA, and mixtures of two or more thereof; preferably 1- or 2-lyso PC, 1- or 2-lyso PE, 1- or 2-lyso PS, 1- or 2-lyso PI, 1- or 2-lyso PA, and mixtures of two or more thereof, and particularly preferably 1- or 2-lyso PC, 1- or 2-lyso PS, and a mixture thereof.

The lipid is a component vital to an organism, and includes an ester bond between an alcohol and a fatty acid. Other than straight chain alcohols, examples of the alcohol include glycerol (glycerin), sterol, and the like. Examples of the fatty acid include various saturated fatty acids or unsaturated fatty acids. Of the lipids, those that have ester bonds between a hydroxyl group of the glycerol, as the alcohol, and a carboxyl group of the fatty acid are referred to as “biological lipids”. Examples of the biological lipids include glycerides and phospholipids.

Examples of the glycerides include triacylglycerols (triglycerides), where all three hydroxyl groups of the glycerol are ester bonded with the fatty acid; diacylglycerols (diglycerides), where two of the three hydroxyl groups of the glycerol are ester bonded with the fatty acid and the other one hydroxyl group is left as-is; and monoacylglycerols (monoglycerides), where one of the three hydroxyl groups of the glycerol is ester bonded with the fatty acid and the other two hydroxyl groups are left as-is.

The phospholipid according to the present invention has a highly unsaturated fatty acid as the fatty acid part. In the present specification, “highly unsaturated fatty acid” refers to a fatty acid having three or more double bonds and having 18 or more, and preferably 20 or more carbon atoms. An n-3 highly unsaturated fatty acid is preferable as the highly unsaturated fatty acid. In the present specification, “n-3 highly unsaturated fatty acid” refers to a fatty acid wherein the third and fourth carbons, counting from the terminal carbon opposite the carboxyl side of the fatty acid molecule, are double bonded. Examples of such a fatty acid include eicosapentaenoic acid (20:5, EPA), docosapentaenoic acid (22:5, DPA), docosahexaenoic acid (22:6, DHA), and the like, preferably EPA and DHA. A percentage of the n-3 highly unsaturated fatty acid occupying the constituent fatty acid of the lipid of the present invention as a fatty acid composition ratio is, for example, from 1 to 100%, preferably from 10 to 90%, and more preferably from 20 to 80%. Because fluidity of the n-3 highly unsaturated fatty acid is high, as greater amounts are included in the lipid, greater effectiveness in providing more beneficial physical characteristics at low temperatures will be achieved. However, at best, unrefined natural materials only contain about 60% of the n-3 highly unsaturated fatty acid and attempting to increase a concentration thereof involves additional costs for concentration.

Any material including a phospholipid such as those described above can be used as the phospholipid of the present invention. Examples of such a material include fish and shellfish extracts, animal extracts, egg yolk extract, plant extracts, fungi (algae) extracts, and the like, specifically, krill oil, fish oil, fish extract, squid extract, bonito ovary extract, animal extract or egg yolk extracts of an animal given a feed compounded with n-3 highly unsaturated fatty acid, flaxseed oil, extracts of genetically modified plants, and the like, and extracts and the like of labyrinthulea. Examples of materials that include a particularly large amount of the phospholipid include krill oil, squid extract, and bonito ovary extract. By using concentrating, extracting and/or purifying, compounding and other techniques known conventionally in the art, a lipid concentration in these materials and a purity can be regulated as desired. For example, by appropriately compounding krill oil, fish oil, flaxseed oil, soy oil, or perilla oil containing the highly unsaturated fatty acid; and krill oil, plant oil (phospholipid of soy origin, phospholipid of rapeseed origin), animal extract (phospholipid of egg yolk origin), marine extract (phospholipid of squid extract origin, phospholipid of fish extract origin, phospholipid of krill extract origin), or the like containing the phospholipid, a phospholipid including the highly unsaturated fatty acid at high concentrations can be produced. In one aspect of the present invention, refined krill oil can be used as the active ingredient.

The orally ingested phospholipid is hydrolyzed into a free fatty acid and a lysoacylglycerophospholipid, a phosphatidic acid, or a lysophosphatidic acid. These hydrolyzates are dissolved by bile acid and by the forming of bile acid micelles. Small intestine epithelial cells incorporate the hydrolyzates from the bile acid micelles and triacylglycerols and diacylglycerophospholipids are resynthesized from the incorporated hydrolyzates. Thus, when the free highly unsaturated fatty acids are ingested by an organism, they are incorporated into the small intestine epithelial cells via bile acid and micelle formation and bond with the glycerol and/or phosphates in the organism. Thereby, they are incorporated as constituent fatty acids of triacylglycerols and/or diacylglycerophospholipids. Therefore, by ingesting the phospholipid or the triacylglycerol together with the highly unsaturated fatty acid, the percentage of phospholipids including highly unsaturated fatty acids among the phospholipids or the triacylglycerol resynthesized in the organism can be increased, and a more excellent brain atrophy preventing effect can be obtained.

For example, when ingesting the phospholipid together with the highly unsaturated fatty acid, a lipid that is appropriately compounded with a fat/oil including both may be used. Alternately, a phospholipid including a highly unsaturated fatty acid as a constituent fatty acid may be used. From the perspectives of ease of absorption, efficacy of use, substance stability, and ease of quality control, the phospholipid including the highly unsaturated fatty acid as the constituent fatty acid is particularly preferable. For example, when ingesting the triacylglycerol together with the highly unsaturated fatty acid, a lipid that is appropriately compounded with a fat/oil including both may be used. Alternately, a triacylglycerol including a highly unsaturated fatty acid as a constituent fatty acid may be used. From the perspectives of ease of absorption, substance stability, and ease of quality control, the triacylglycerol including the highly unsaturated fatty acid as the constituent fatty acid is particularly preferable.

The brain atrophy prevention agent of the present invention may also include other components included in krill oil, such as, for example, astaxanthin, sterol, and the like. Astaxanthin is a compound belonging to carotenoids commonly found in crustacea such as crabs and shrimp. The astaxanthin may be present in a free state or may be present in a lipid state via ester bonding. Additionally, from 1 to 10,000 ppm, preferably from 5 to 5,000 ppm, and more preferably from 10 to 1,000 ppm of the astaxanthin in a free state may be separately added to the brain atrophy prevention agent. The astaxanthin, as an endogenous antioxidant, contributes to the stability of the highly unsaturated fatty acid, and, thus, is preferably included in abundance. However, if too much of the astaxanthin is included, problems with color and taste will easily occur. The sterol contributes to the fluidity of the phospholipid and also contributes to the absorption of the brain atrophy prevention agent of the present invention.

In the present specification, it is sufficient that the “krill” be an arthropod belonging to the phylum Arthropoda, subphylum Crustacea, class Malacostraca and includes arthropods belonging to the phylum Arthropoda, subphylum Crustacea, class Malacostraca, order Eucarida, family Euphausiacea such as, for example, Antarctic krills (Euphausia superba), and arthropods belonging to the phylum Arthropoda, subphylum Crustacea, class Malacostraca, order Euphausiacea, family Euphausiidae such as, for example, Mysidacea caught in the seas around Japan, and the like. However, from the perspective of stability of catch volume and uniformity of the lipid component, Antarctic krills are particularly preferable. In the present specification, “lipid of krill origin” refers to a lipid obtained from the Antarctic krills described above.

The phospholipid of krill origin used in the present invention can be acquired by a known method of manufacturing. For example, the phospholipid can be produced while referring to the known methods described in WO2000/023546A1, WO2009/027692A1, WO2010/035749A1, WO2010/035750A1, or the like. At the least, the phospholipid that can be produced via the methods described in the international publications can be preferably used in the brain atrophy prevention agent of the present invention.

In the brain atrophy prevention agent of the present invention, the phospholipid can be obtained by, for example, following a method described in the international publications mentioned above, and using an appropriate organic solvent to extract the phospholipid from a solid content originating from a source material of krills. Appropriate examples of the organic solvent include alcohols such as methanol, ethanol, propanol, isopropanol, butanol, propylene glycol, butylene glycol; methyl acetate, ethyl acetate, acetone, chloroform, toluene, pentane, hexane, cyclohexane, and the like. These may be used alone or in combinations of two or more. In such cases, a mixture ratio of the solvent or a ratio of the source material to the solvent can be set as desired.

The solid content originating from a source material of krills can be obtained, for examples, by obtaining a squeezed fluid by squeezing all or a part of dried, milled, raw, or frozen krills; and separating the solid content and a water soluble component by heating the squeezed fluid. For the squeezing, a commonly used apparatus can be used. For example, a hydraulic press, a screw press, a meat and bone separator, a press dehydrator, a centrifuge, and the like, or a combination thereof can be used.

The squeezed fluid may be heated under atmospheric pressure, pressurized, or reduced pressure conditions to 50° C. or higher, and preferably to from 70 to 150° C., and particularly preferably to from 85 to 110° C. Through this heating, the solid content (thermal coagulum) and the water soluble component are separated, and through filtering, centrifuging, or the like, a thermal coagulum is obtained. Furthermore, the thermal coagulum can be appropriately dried and used. The drying can be performed by any one or a combination of hot air drying, drying using steam, drying by high frequency/microwave heating, vacuum/reduced pressure drying, drying by freezing and thawing, and drying using a drying agent. Because oxidized lipids cause unpleasant odors if the temperature is too high during the drying process, the drying should be carried out at 90° C. or lower, preferably 75° C. or lower, and more preferably 55° C. or lower. Drying is preferable because volatile impurities are removed thereby. The thermal coagulum or the dried product thereof includes astaxanthin, and therefore can be preferably used as the brain atrophy prevention agent of the present invention.

Namely, it is preferred to use in the present invention a krill oil that is obtainable by a process comprising: obtaining a squeezed liquid by squeezing a whole krill or a part thereof, heating the squeezed liquid to a temperature at which proteins contained in the squeezed liquid coagulate, carrying out solid-liquid separation so as to separate the heated squeezed liquid into a solid component that contains lipid components and an aqueous component that contains water-soluble components, washing the resulting solid containing lipids or a dried product thereof with water, dehydrating and/or drying, and then extracting lipids from the solid containing lipids or the dried product thereof, or a phosphatidylserine-containing oil/fat produced with the krill oil as a raw material.

A krill oil obtained by the process is characterized in a low content of free fatty acids and the like, particularly a low content of lysophospholipids. In this regard, a krill oil suitable to use in the present invention contains 90 wt % or more of diacylglycerophospholipids and 6 wt % or less of lysoacylglycerophospholipids in the phospholipid composition thereof. Preferably, a krill oil contains 95 wt % or more of diacylglycerophospholipids and 3 wt % or less of lysoacylglycerophospholipids in the phospholipid composition thereof. More preferably, a krill oil contains 97 wt % or more of diacylglycerophospholipid and 2 wt % or less of lysoacylglycerophospholipids in the phospholipid composition thereof.

Further, it is preferred that a krill oil in the present invention contains 40 wt % or more of phospholipids; acid number thereof is 20 or less, preferably 10 or less, more preferably 5 or less; peroxide value (POV) thereof is 5 or less, preferably 3 or less, more preferably 1 or less; a ratio of n-3 highly unsaturated fatty acid in the total fatty acid is 20 wt % or more, preferably 25 wt % or more; and the krill oil contains astaxanthin of 150 ppm or more, preferably 200 ppm or more.

Generally, a purification method of phospholipid wherein an amount of residue of the impurities is small is preferred. The thermal coagulum of the squeezed krill fluid or the dried product thereof is fit for such a purpose because a concentration of the water soluble component thereof can be reduced by washing with water. The washing with water can be performed using an amount of freshwater or saltwater 4-times, and preferably 10-times or more the amount of the dry content weight in the thermal coagulum or the dried product thereof. The washing with water is preferably carried out at least twice, and more preferably at least three times. The washing with water can be performed by adding water to a container in which the thermal coagulum or the dried product thereof has been placed, and then separating the moisture content after waiting for 5 minutes or longer. Depending on a shape of the thermal coagulum or the dried product thereof, a sufficient amount of agitation can also be effective. Additionally, the washing with water can be performed by washing the thermal coagulum or the dried product thereof in a container with running water.

Furthermore, for example, by treating the thermal coagulum or the dried product thereof, or a washed product thereof as described below, a fraction including a greater amount of the PC can be obtained. For example, an extract oil is obtained by treating the thermal coagulum or the dried product thereof, or the washed product thereof with a solvent such as ethanol, hexane, chloroform, acetone, or the like. Next, impurities and the phospholipid fraction are separated by subjecting the extract oil to chromatography using silica gel or the like, and the phospholipid fraction is concentrated. The fraction is rich in PC.

In one aspect of the present invention, a product of enzyme reaction with a krill oil as a substrate can be used as the active ingredient. The enzyme reaction can be an enzyme reaction that converts the phosphatidylcholine that is included in krill oil to another phospholipid, such as phosphatidylserine, phosphatidylethanolamine, phosphatidic acid, phosphatidylglycerol, phosphatidylinositol, and the like.

For example, the PS can be obtained by enzymatically reacting PC and serine using the catalytic action of phospholipase D. An amount of the serine used with respect to an amount of the phospholipid used in the reaction can be set to from 0.5 to 3 weight ratio, and preferably from 1 to 2 weight ratio. The phospholipase D can be used at from 0.05 to 0.2 weight ratio, and preferably from 0.1 to 0.15 weight ratio per 1 g of the phospholipid. The phospholipase D that can be used include those originating from microorganisms and vegetables such as cabbage and the like.

The enzymatic reaction can be performed using a method known in the art. For example, the enzymatic reaction can be performed in a solvent such as ethyl acetate and the like at from 35 to 45° C. for from 20 to 24 hours.

The PS used in the present invention can be obtained by extraction from animal tissue.

The brain atrophy preventing effect of the brain atrophy prevention agent of the present invention can be confirmed by imaging tests such as CT, MRI, and PET, and the like. Furthermore, the effect can be directly confirmed by performing animal testing, extracting the whole brain, and measuring the brain weight. Furthermore, along with the effect of improving brain atrophy of the present invention, the effects of improving memory capability and learning capability and the like as well as improving the symptoms of Alzheimer's disease and suppressing the progression of symptoms can also be confirmed. Furthermore, the effect of preventing brain atrophy of the present invention can also be confirmed by evaluation of the brain antioxidant capacity by measuring the expression of SOD (superoxide dismutase) activity, GSH-Px (glutathione peroxidase) activity, and MDA concentration, by measuring the level of expression of Iba-1 (ionized calcium binding adapter molecule 1) as a marker for microglia cells, and measuring the expression of IGF-1 (insulin-like growth factor 1) as a marker for brain aging.

The present invention can be used for treating or preventing diseases related to brain atrophy. Diseases related to brain atrophy include Alzheimer's disease, amnesia, memory impairment, mobility impairment, and the like.

The brain atrophy prevention agent of the present invention can be used in combination with other components which are commonly known brain function improving effects, such as vitamins (e.g. vitamin B6, vitamin C), Gamma-amino butyric acid (GABA), astaxanthin, arachidonic acid, fish oil, lecithin, natural polyphenols (such as ginkgo biloba leaf extract) and the like, as necessary. The brain atrophy prevention agent of the present invention may include components such as conventionally known colorants, preservatives, perfumes, flavorants, coating agents, antioxidants, vitamins, amino acids, peptides, proteins, minerals (i.e. iron, zinc, magnesium, iodine, etc.) and the like, as necessary.

Examples of the antioxidant includes tocopherol, dried yeast, glutathione, lipoic acid, quercetin, catechin, coenzyme Q10, enzogenol, proanthocyanidins, anthocyanidin, anthocyanin, carotenes, lycopene, flavonoid, resveratrol, isoflavones, zinc, melatonin, ginkgo leaf, Alpinia zerumbet leaf, hibiscus, or extracts thereof.

Examples of the vitamin include the vitamin A group (i.e. retinal, retinol, retinoic acid, carotene, dehydroretinal, lycopene, and salts thereof); the vitamin B group (i.e. thiamin, thiamin disulfide, dicethiamine, octotiamine, cycotiamine, bisibuthiamine, bisbentiamine, prosultiamine, benfotiamine, fursultiamine, riboflavin, flavin adenine dinucleotide, pyridoxine, pyridoxal, hydroxocobalamin, cyanocobalamin, methylcobalamin, deoxyadenocobalamin, folic acid, tetrahydro folic acid, dihydro folic acid, nicotinic acid, nicotinic acid amide, nicotinic alcohol, pantothenic acid, panthenol, biotin, choline, inositol, pangamic acid, and salts thereof); the vitamin C group (i.e. ascorbic acid and derivatives thereof, erythorbic acid and derivatives thereof, and salts thereof that are pharmacologically acceptable); the vitamin D group (i.e. ergocalciferol, cholecalciferol, hydroxycholecalciferol, dihydroxycholecalciferol, dihydrotachysterol, and salts thereof that are pharmacologically acceptable); the vitamin E group (i.e. tocopherol and derivatives thereof, ubiquinone derivatives, and salts thereof that are pharmacologically acceptable); and other vitamins (i.e. carnitine, ferulic acid, γ-oryzanol, orotic acid, rutin (vitamin P), eriocitrin, hesperidin, and salts thereof that are pharmacologically acceptable).

Examples of the amino acid include leucine, isoleucine, valine, methionine, threonine, alanine, phenylalanine, tryptophan, lysine, glycine, asparagine, aspartic acid, serine, glutamine, glutamic acid, proline, tyrosine, cysteine, histidine, ornithine, hydroxyproline, hydroxylysine, glycylglycine, aminoethylsulfonic acid (taurine), cystine, and salts thereof that are pharmacologically acceptable.

The brain atrophy prevention agent of the present invention may be prepared in the form of a pharmaceutical composition, functional food, health food, supplement, or the like such as, for example, various solid formulations such as granule formulations (including dry syrups), capsule formulations (soft capsules and hard capsules), tablet formulations (including chewable tablets and the like), powdered formulations (powders), pill formulations, and the like; or liquid formulations such as liquid formulations for internal use (including liquid formulations, suspension formulations, syrup formulations, etc.) and the like.

Examples of additives that help with formulation include excipients, lubricants, binders, disintegrating agents, fluidization agents, dispersing agents, wetting agents, preservatives, thickening agents, pH adjusting agents, colorants, corrigents, surfactants, and solubilization agents. Additionally, prepared as a liquid formulation, thickening agents such as pectin, xanthan gum, guar gum, and the like can be compounded. Moreover, the brain atrophy prevention agent of the present invention can be formed into a coated tablet formulation by using a coating agent, or be formed into a paste-like gelatin formulation. Furthermore, even when preparing the brain atrophy prevention agent in other forms, it is sufficient to follow conventional methods.

Furthermore, the brain atrophy prevention agent of the present invention can be used as various foods and drinks such as, for example, beverages, confectioneries, breads, soups, and the like, or as an added component thereof. Methods of manufacturing these foods and drinks are not particularly limited as long as the effectiveness of the present invention is not hindered, and it is sufficient that a process used by a person skilled in the art for each application be followed.

Furthermore, the brain atrophy prevention agent of the present invention can be used as a feed for animals, other than humans, or as an added component thereof. The brain atrophy prevention agent of the present invention may be compounded with the animal feed that is normally administered to each animal, and can be used regardless of the form of the animal feed, such as mash, flakes, crumble, powder, granules, moist pellets, dry pellets, EP pellets, or the like. Methods of manufacturing these animal feeds are not particularly limited as long as the effectiveness of the present invention is not hindered, and it is sufficient that a process used by a person skilled in the art for each application be followed.

### EXAMPLES

The present invention is described in detail by means of the examples shown below, but the scope of the present invention is not limited thereto.

### Example 1

Production of Phosphatidylcholine

A squeezed fluid (3 tons) was obtained by squeezing Antarctic krills (10 tons) caught in the Antarctic Ocean from February to November 2009 in a meat and bone separator (BAADER 605, manufactured by Baader) immediately after being caught. This squeezed fluid was transferred to a stainless steel tank 800 kg at a time, and was heated by directly injecting water vapor of a temperature of 140° C. After heating for approximately 60 minutes, it was confirmed that the temperature had reached 85° C., and the heating was then stopped. A valve in the bottom of the tank was opened, the liquid component was removed by being allowed to pass through a mesh having an aperture size of 2 mm by means of gravity, the solid component (thermal coagulum) was washed by being showered with an equal quantity of water, and 12 kg batches of the thermal coagulum were placed in aluminum trays and rapidly frozen using a contact freezer. A total weight of the obtained coagulum was 2.25 tons.

A frozen product (1 ton) was introduced into water (3,000 liters) and heated while stirring until a temperature reached 65° C., and was held at 65° C. for 10 minutes. The water was removed via 24 mesh nylon, and the solid component was placed in 3,000 liters of water (at 20° C.). After stirring for 15 minutes, the mixture was strained using a 24 mesh nylon. Then, the strained mixture was placed in a dewatering centrifuge (Centrifuge 0-30, manufactured by Tanabe Willtec Inc.; 15 seconds), and a solid content was obtained having a moisture content of approximately 73%. 0.3% of tocopherol was added to this solid content. The resulting mixture was blended thoroughly using a mixer, dried for 3.2 hours by means of hot air drying at a temperature of from 70 to 75° C., and a cleaned and dried product (170 kg) was obtained. Other frozen products were processed in the same manner.

99% of ethanol (1,200 liters) was added to the cleaned and dried product (300 kg), and the resulting mixture was heated to 60° C. and mixed for two hours. Thereafter, a liquid extract A and a lees extract A were obtained by solid-liquid separation via natural dripping using a 100 mesh nylon. 99% of ethanol (800 liters) was added to the lees extract A. After heating to 60° C. and mixing for two hours, the resulting mixture was solid-liquid separated using a 100 mesh nylon, and a liquid extract B and a lees extract B were obtained. 99% of ethanol (700 liters) was added to the lees extract B. After heating to 60° C. and mixing for two hours, the resulting mixture was solid-liquid separated using a 100 mesh nylon, and a liquid extract C and a lees extract C were obtained. When the extraction liquid A, extraction liquid B, and extraction liquid C were combined, the total weight thereof was 2,021 kg. This combination was concentrated in vacuo at a temperature of 60° C. or less, the ethanol and water were removed, and extracted lipids (145.0 kg) were obtained. Components of the obtained extracted lipids and a composition of the fatty acid are shown in Table 1 and Table 2.

TABLE 1

| | | Extracted Lipids |
|---|---|---|
| Water | (%) | 0.48 |
| Ethanol | (%) | 0.42 |
| Phospholipid | (%) | 46.9 |
| Acid Number | | 4.3 |
| Peroxide Value | (meq/kg) | 0.1 |
| Astaxanthin | (ppm) | 343 |

TABLE 2

| | | Extracted Lipids |
|---|---|---|
| Fatty Acid | C14:0 | 11.8 |
| Composition | C16:0 | 22.6 |
| (%) | C18:1 | 18.5 |
| | C18:2 | 1.5 |
| | C18:3 | 0.8 |
| | C18:4 | 2.3 |
| | EPA | 14.8 |
| | DHA | 6.9 |

The extracted lipids were adsorbed on a silica gel (Microsphere gel manufactured by Asahi Glass Co., Ltd.; Model: M.S. GEL SIL; 300 g) column. After adding chloroform to the column and rinsing off the neutral lipids, a phospholipid fraction (0.228 g) was collected by adding methanol. A lipid content in 10 g of the dried product of the thermal coagulum was 4.72 g.

The lipid component was separated by subjecting the phospholipid fraction to thin layer chromatography using a developing solvent containing chloroform, methanol, and water at a ratio of 65:25:4. Lipid components were quantitatively analyzed using a thin layer automatic detecting device (Model: Iatroscan™ MK-6, manufactured by Mitsubishi Kagaku Iatron, Inc.). As a result, it was discovered that the phospholipid fraction included phosphatidylcholine (96 wt %) and phosphatidylethanolamine (4 wt %).

The fatty acid in the phospholipid fraction was methyl-esterized in boron trifluoride and was subjected to gas chromatography set to the following parameters. Thereby, a fatty acid composition was analyzed.

Gas chromatography: Model: 6890N, manufactured by Agilent Technologies

Column: DB-WAX, (Model 122-7032, manufactured by J&W Scientific)

Carrier gas: Helium

Detector: Hydrogen ionization detector

The results of the analysis are shown below in Table 3.

TABLE 3

| | Content in the composition |
|---|---|
| PC content | 96 wt %. |
| EPA content | 29.7 wt % |
| DHA content | 12.1 wt % |

### Example 2

Production of a Phosphatidylserine-Containing Composition

After adding L-serine (200 g) to a sodium acetate buffer (pH 5.6, 400 ml) and then adding phospholipase D (4000 unit/g, 2 g) of actinomycete origin, the serine was completely dissolved by mixing at 40° C. The solution was combined with astaxanthin (340 ppm) and ethyl acetate (500 ml) in which a phospholipid of krill origin (PC30; 100 g) containing PC (35 wt %) was dissolved; and reacted for 24 hours at 40° C. while mixing at 200 rpm.

After completion of the reacting, the reaction solution was allowed to stand and a separated top layer was collected. The top layer was washed three times with water in order to remove the residual serine and enzyme. By concentrating the solvent layer, a composition (85.2 g, yield with respect to the phospholipid: 85.2%) containing PS was obtained. Results of analyzing the content of the PS, EPA, and DHA in the composition are shown below in Table 4.

TABLE 4

| | Content in the composition |
|---|---|
| PS content | 30.5 wt % |
| EPA content | 15.4 wt % |
| DHA content | 7.0 wt % |

### Example 3

Production of a Phosphatidylserine-Containing Composition

After adding L-serine (200 g) to a sodium acetate buffer (pH 5.6, 400 ml) and then adding phospholipase D (4000 unit/g, 2 g) of actinomycete origin, the serine was completely dissolved by mixing at 40° C. The solution was combined with astaxanthin (450 ppm) and ethyl acetate (500 ml) in which a phospholipid of krill origin (PC45; 100 g) containing PC (45 wt %) was dissolved; and reacted for 24 hours at 40° C. while mixing at 200 rpm.

After completion of the reacting, the reaction solution was allowed to stand and a separated top layer was collected. The top layer was washed three times with water in order to remove the residual serine and enzyme. By concentrating the solvent layer, a PS-containing composition (85.9 g, yield with respect to the phospholipid: 85.9%) was obtained. Results of analyzing the content of the PS, EPA, and DHA in the composition are shown below in Table 5.

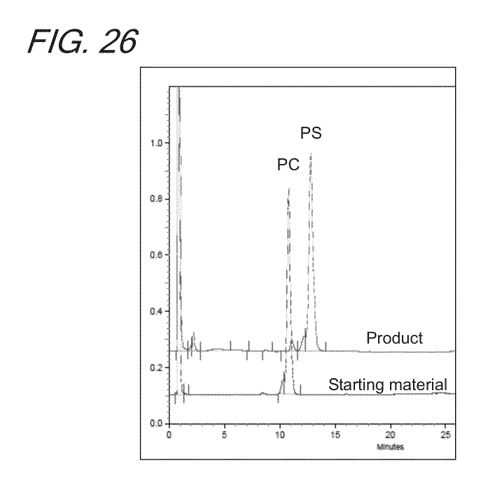

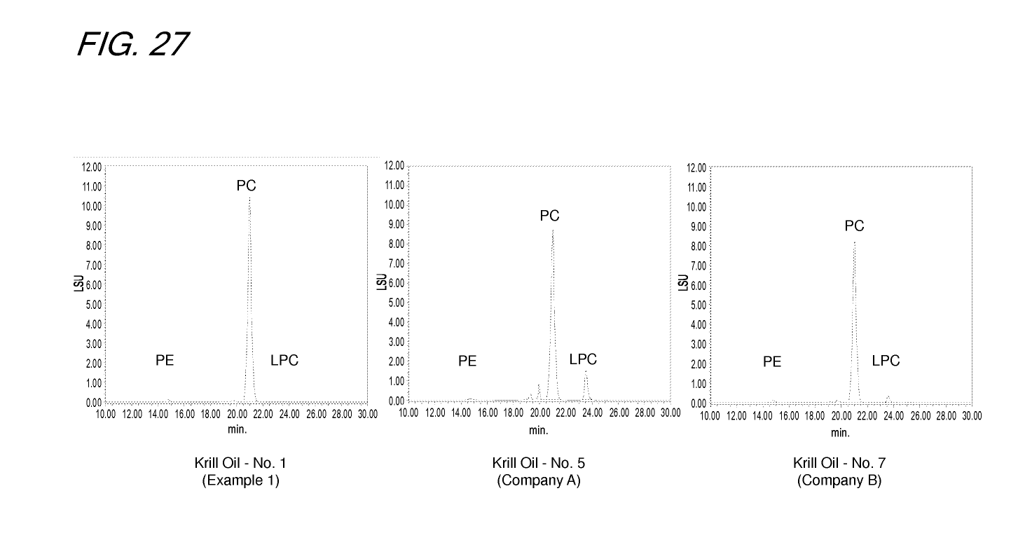

A phospholipid composition of the starting material and the product of the reaction was measured by high performance chromatography under the following condition. The results are shown in FIG. 26. It was confirmed that PC was converted to PS without increase of lysophospholipid.

Condition of HPLC Analysis

1. Analytical instrument: Jasco LCSS-905 (JASCO Corporation)

2. Column: Develosil 60-5, I.D. 4.6×150 mm

3. Column temperature: 40° C.

4. Mobile phase: A: Chloroform
B: Methanol/Water (95:5, vol/vol)

5. Flow rate: 1.0 mL/min

6. Injection volume: 5 μL

7. Detector: 3300 ELSD (Alltech)
Drift tube temperature: 50° C.
   Nebulizer temperature: 30° C.
   Gas: Nitrogen

8. Gradient system:

| Time(min) | A % | B % |
|---|---|---|
| 0 | 100 | 0 |
| 10 | 0 | 100 |
| 30 | 0 | 10 |

TABLE 5

| | Content in the composition |
|---|---|
| PS | 40.0 wt % |
| EPA | 32.0 wt % |
| DHA | 14.4 wt % |

### Comparative Example 1

To confirm the properties of extracted lipids obtained by the process described in Example 1, comparative analysis tests with krill oils produced by the other companies were conducted. For 4 lots of the extracted lipids obtained by the process of Example 1 and 2 krill oil products of other company, acid value was measured by the procedure described in AOAC 969.17, a phospholipid composition was measured by high performance liquid chromatography (HPLC) under the following condition. The results are shown in Table 6.

HPLC Analysis Condition

1. Analytical instrument: Alliance e2695 (Waters)

2. Column: Sun Fire Silica 5 μm, I.D. 4.6×150 mm

3. Column temperature: 45° C.

4. Mobile phase: A: Chloroform
B: Methanol/Water (95:5, vol/vol)

5. Flow rate: 1.0 mL/min

6. Injection volume: 5 μL

7. Detector: 2424 ELSD
Drift tube temperature: 50° C.
   Nebulizer temperature: 30° C.
   Gas: Nitrogen

8. Gradient system:

| Time(min) | A % | B % |
|---|---|---|
| 0 | 99 | 1 |
| 15 | 75 | 25 |
| 20 | 10 | 90 |
| 30 | 10 | 90 |
| 35 | 99 | 1 |
| 40 | 99 | 1 |

TABLE 6

| | | Acid | HPLC area ratio % | | | |
|---|---|---|---|---|---|---|
| Sample | No. | value | PE | PC | LPC | PC/LPC |
| Extracted lipids | 1 | 4.3 | 0.86 | 11.57 | 0.25 | 46.28 |
| obtainable by the | 2 | 4.4 | 0.92 | 12.25 | 0.19 | 64.47 |
| procedure of | 3 | 4.7 | 0.89 | 11.71 | 0.21 | 55.76 |
| Example 1 | 4 | 4.5 | 0.84 | 11.74 | NT | — |
| Krill Oil | 5 | 15.1 | 0.69 | 9.03 | 3.72 | 2.43 |
| (Company A) | 6 | 13.4 | 0.70 | 9.98 | 3.57 | 2.80 |
| Krill Oil | 7 | 25.5 | 0.92 | 10.07 | 1.31 | 7.69 |
| (Company B) | | | | | | |

### Test Example 1

Brain Atrophy Preventing Test

A brain atrophy preventing and brain function improving test of phospholipids was performed using Senescence-accelerated mouse P10 (SAMP10) that was identified as a brain atrophy model animal. The effect of a composition containing phosphatidylcholine derived from krill and a composition containing phosphatidylserine were evaluated.

(1) Test Animal

In the test, SPF animals (Specific Pathogen Free animals, SPF) procured from First Teaching Hospital of Tianjin University of Traditional Chinese Medicine (SCXK2008-0001, No. 0001375, Tianjin, China) were used. SAMP10 (2 month old, male) animals for the control group and each administration group and SAMR1 (for base group, 2 month old, male) were procured and raised under the following conditions: temperature 23±2° C., humidity 50±10%, artificial lighting switching every 12 hours between light and dark, feed for SAM, and sterilized water, with 5 or 10 animal/cage.

After acclimatizing for 7 days, each animal was randomly grouped as shown below.

SAMR1 group: 12 animals/group

SAMP10 group: 15 animals/group (after testing, the number of surviving animals in each group was: 10 to 13 animals).

Note, the characteristics of SAMP10 which were brain atrophy model animals are referenced in the following documentation.

1. A Shimada, Age-dependent cerebral atrophy and cognitive dysfunction in SAMP10 mice, Neurobiology of Aging, 20:125-136, 1999;

2. A. Shimada, M. Hosokawa, et al, Localization of atrophy-prone areas in the aging mouse brain: comparison between the brain atrophy model SAM-P/10 and the normal control SAM-R/1, Neuroscience, 59(4): 859-869, 1994;

3. A. Shimada, A. Ohta, et al, Inbred SAM-P/10 as a Mouse Model of Spontaneous, Inherited Brain Atrophy, J of Neuropath and Exper Neurology, 51(4): 440-450, 1992;

3. A. Shimada, H. Keino, et al, Age-related Loss of Synapses in the Frontal Contex of SAMP10 mouse: A Model of Cerebral Degeneration, SYNAPSE, 48: 198-204, 2003;

4. A. Shimada, H. Keino, et al, Age-related Progressive Neuronal DNA Damage Associates With Cerebral Degeneration in a Mouse Model of Accelerated Senescence, J of Gerontology: BIOLOGICAL SCIENCE, 57A: B415-B421, 2002;

5. T. Sasaki, K. Unno, et al, Age-related increase of superoxide generation in the brains of mammals and birds, Aging Cell, 7: 459-469, 2008;

6. A. Shimada, A. Ohta, et al, Age-related deterioration in conditional task in the SAM-P/10 mouse, an animal model of spontaneous brain atrophy, Brain Research, 608: 266-272, 1993.

(2) Testing Process

The following substances were used as the administration samples: Phosphatidylcholine derived from krill produced in working example 1 (Krill Phosphatidylcholine, K-PC: 40 wt %).

Phosphatidylserine derived from Krill produced in working example 3 (Krill Phosphatidylserine, K-PS:40 wt %).

Physiological saline solution (for comparison group)

The administration samples were provided by oral administration one time per day for 45 days or for 90 days, continuously. The administration groups and the administration dose were as shown below.

SAMR1 group: SAMR1, physiological saline solution 0.2 mL/animal administration (45 days administration)

Cont. group: SAMP10, physiological saline solution 0.2 mL/animal administration (45 days and 90 days administration)

PC group: SAMP 10, K-PC 0 2 mL/animal administration (45 days and 90 days administration).

PS group: SAMP10, K-PS 0 2 mL/animal administration (45 days and 90 days administration).

PC+PS: SAMP10, K-PC 0.1 mL/animal administration and K-PS 0 1 mL/animal administration (45 days and 90 days administration).

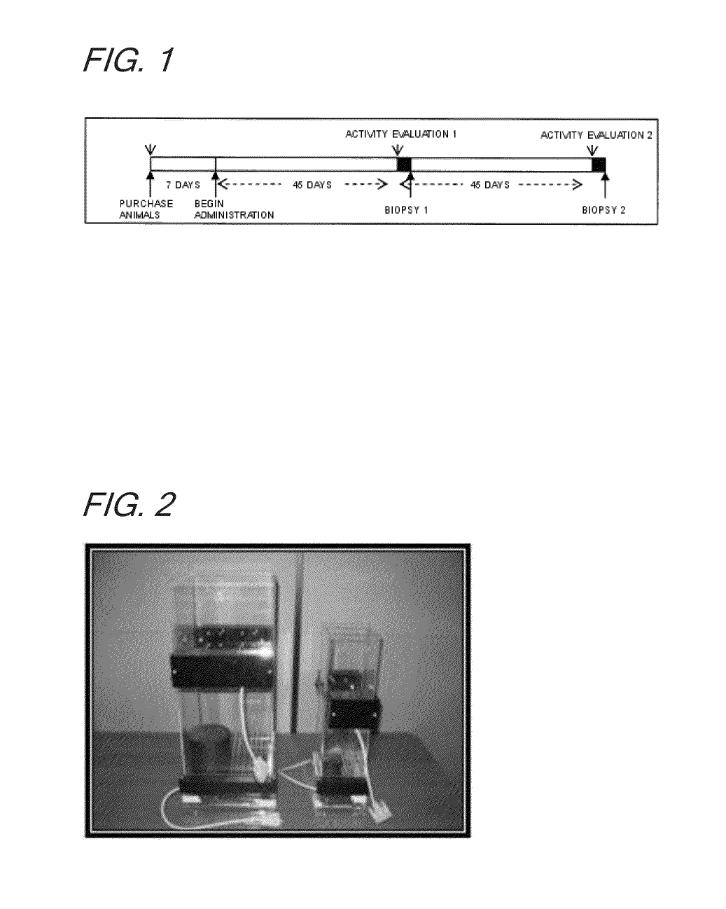

Behavior testing was performed as described below on the last day of administration, and the animal was chemically euthanized the following day, and the organs were extracted for testing. The test results are shown in FIG. 1.

(3) Step Down Test

An animal was placed in a test device (FIG. 2), and acclimatize for 3 minutes, and then the animal was placed on the bottom level (electrocution level), a current with 36 V was immediately applied to provide an electric shock to the feet of the animal. Testing was started from the moment the electrically shocked animals escaped to the upper level (safe level) by using an inherent escape behavior. The test evaluates the memory capability by recording the number of times that the animal dropped from the upper level to the lower level (error count) and the time residing in the lower level (error time) during the 5 minutes testing period.

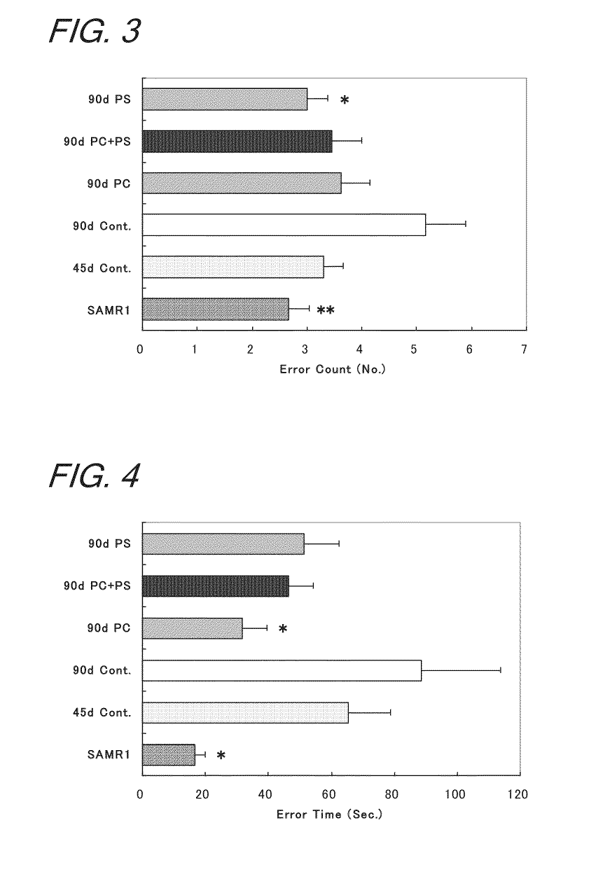

The test results are shown as the number of errors in FIG. 3, and the error time in FIG. 4. When the number of step down test errors of each group were compared in FIG. 3, the 90 day Cont. group had a much higher increase in the number of errors as compared to the SAMR1 group and the 45 day Cont. group, thus demonstrating a decline in memory capability due to aging. The PS group demonstrated significantly improved results compared to the 90 day Cont. group, and the PC and PC+PS groups demonstrated an improvement tendency.

When the step down test error time of each group was compared in FIG. 4, the 90 day Cont. groups had a much higher increase in the error time as compared to the SAMR1 group, thus demonstrating a decline in memory capability due to aging. The PC group demonstrated significantly improved results compared to the 90 day Cont. group, and the PS and PC+PS groups demonstrated an improvement tendency.

(4) Y Maze Test

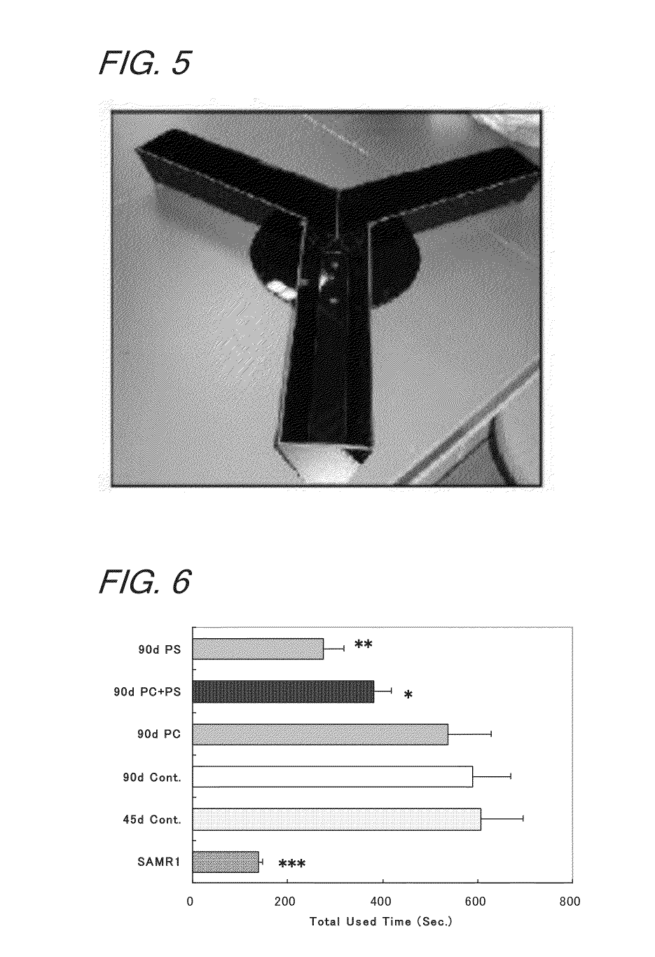

The animal was placed in the test device (FIG. 5) and acclimatized for 3 to 5 minutes as confirmed by repeatedly entering and leaving the arms. During this time, a light was on in order to allow confirmation that the residing area was safe, and a light was turned on in an arm without electric shock in order to confirm that the arm where the light was on was a safe area. Next, beginning at the arm where the animal resided, the test was started by applying a current of 30 to 40 V in order to provide an electric shock to the feet of the animal for 5 seconds. The test was configured such that current was always applied by manual operation to two of the three arms, while the remaining arm was a safe region where the light was ON. The electrically shocked animal implemented innate avoidance activity, and would attempt to escape into the other arms. For the animals that entered the safe region, the lighting time was continued for 5 seconds, and then switched to electrification such that this arm became a starting point where the next electrical shock was applied. In the test, the steps of applying electrical shock followed by escape activity were repeated. The time until the animal entered the safe region nine times in a row from 10 electrical shocks and implemented accurate avoidance activity was recorded as the total used time.

The test results are shown in FIG. 6. When the total time for the Y maze test was compared for each of the 90 day groups, the Cont. groups had greatly increased total use time as compared to the SAMR1 group, thus demonstrating a loss of memory capability due to aging. The PC+PS and PS groups demonstrated significantly improved results compared to the 90 day Cont. group.

(5) Brain Weight

The whole brain was removed and accurately dissected vertically and then the left brain and right brain were separated. Next, the half brains were laterally dissected precisely in the middle and separated into a half brain front part and a half brain rear part, the weight of each section was weighed, and the brain weight rate (brain weight/body weight) was calculated.

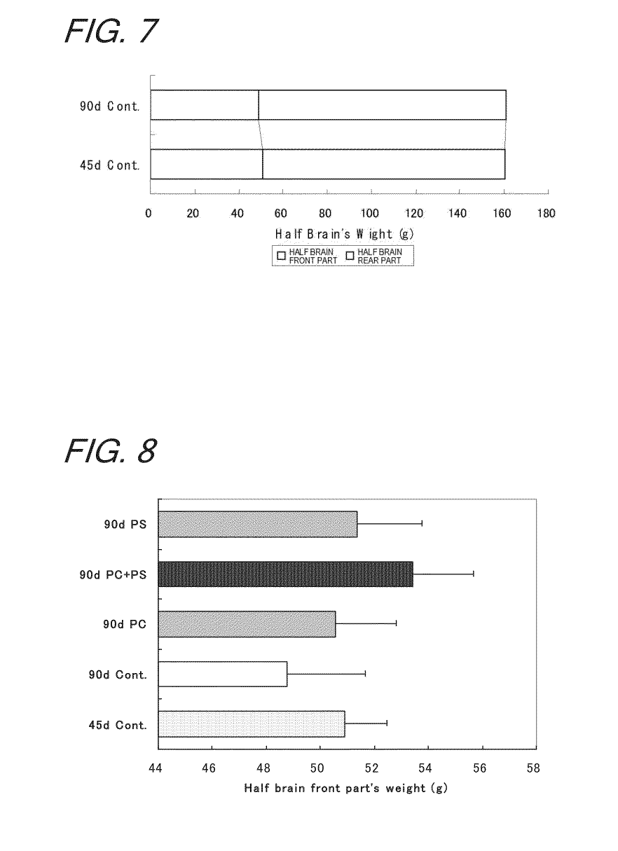

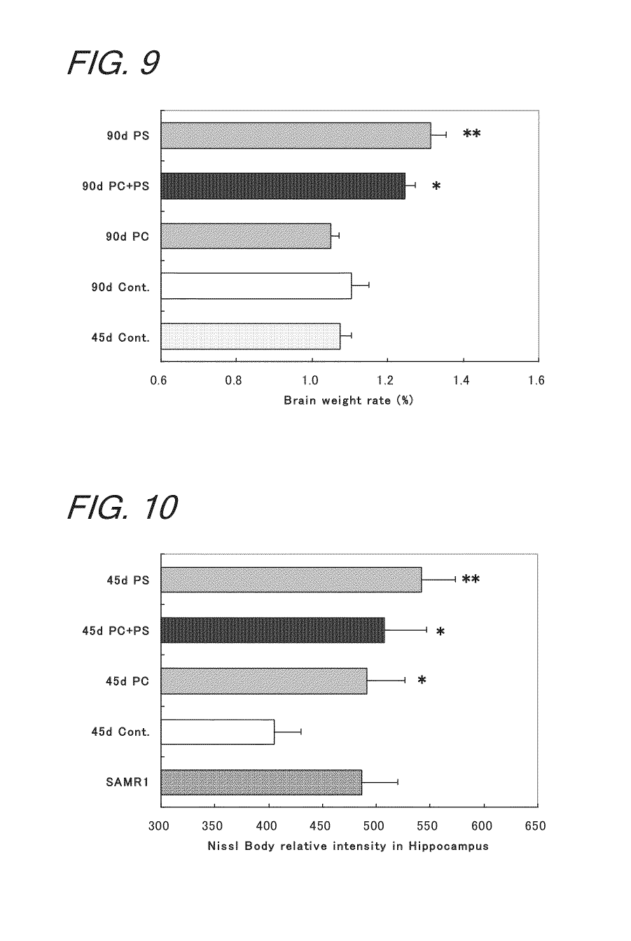

The measurement results are shown in FIG. 7 through FIG. 9. In FIG. 7, when a comparison of the AMP10 animal half brain weights were compared, the half brain weights of the 90 day Cont. group did not change significantly as compared to the 45 Cont. group, but on the other hand, the half brain front part weight demonstrated a tendency to be lower. Thus, brain atrophy phenomenon due to aging was confirmed. In FIG. 8, when the 90 day half brain front part weights were compared, the PC administration group, PC+PS administration group, and the PS administration group demonstrated a tendency to increase as compared to the 90 day Cont. group. In FIG. 9, when the total brain weights were compared for each 90 day group, the PC+PS administration group and the PS administration group demonstrated significantly increasing effects as compared to the 90 day Cont. group.

(6) Brain Nerves

The right half brain was fixed using 4% para-formaldehyde solution, water was removed by a standard method, and then paraffin embedding was performed. Next, the embedded brain sample was cut to a thickness of 5 μm, dyed using hematoxylin eosine, and then the Nissl body density of the hippocampus and cerebral cortex regions were determined by measuring the amount of light exposure captured from these areas using a fluorescence microscope (Eclipse 50i, manufactured by Nikon Corporation).

With regards to the density of the Nissl body, a graph of the amount of exposure from the Nissl body of the hippocampus is shown in FIG. 10, and a graph of the amount of exposure from the Nissl body of the cerebral cortex is shown in FIG. 11. When comparing the Nissl body density for the hippocampus of each 45 day group (FIG. 10), the 45 day Cont. group demonstrated a tendency to be lower as compared to the SAMR1 group. Furthermore, a significant improvement effect was demonstrated by each of the administration groups as compared to the 45 day Cont. group. When comparing the Nissl body density for the cerebral cortex of each 90 day group (FIG. 11), the 90 day Cont. group demonstrated a tendency to be lower as compared to the SAMR1 group. Furthermore, the PC+PS administration group and the PS administration group demonstrated significant improvement effects as compared to the 90 day Cont. group, and the PC administration group also showed a tendency for improvement.

The embedded brain sample was cut to a thickness of 5 μm, the paraffin was removed using an alcohol solution, and then the sample was washed using phosphoric acid buffered physiological saline solution (PBS, pH 7.4) and citric acid buffered solution (10 mM sodium citrate, 0.05% Tween 20, pH 6.0), the protein bonds were broken using a 10 μg/mL protease K solution (20 mg/mL, protease K, 1 M Tris-HCl, 0.5 M EDTA, pH 8.0), and then an immunological reaction was performed overnight using IGF-1 antibodies (Anti-rabbit, 1:100, Santa Cruz Biotechnology, USA) by adding 50 μL of goat blood at 4° C. Next, the immunological reaction solution was washed with phosphoric acid buffer solution, colored using 3,3′-diaminobenzidine (DAB), and then the intensity of an IGF-1 (insulin-like growth factor 1) positive reaction was measured using a fluorescence microscope.

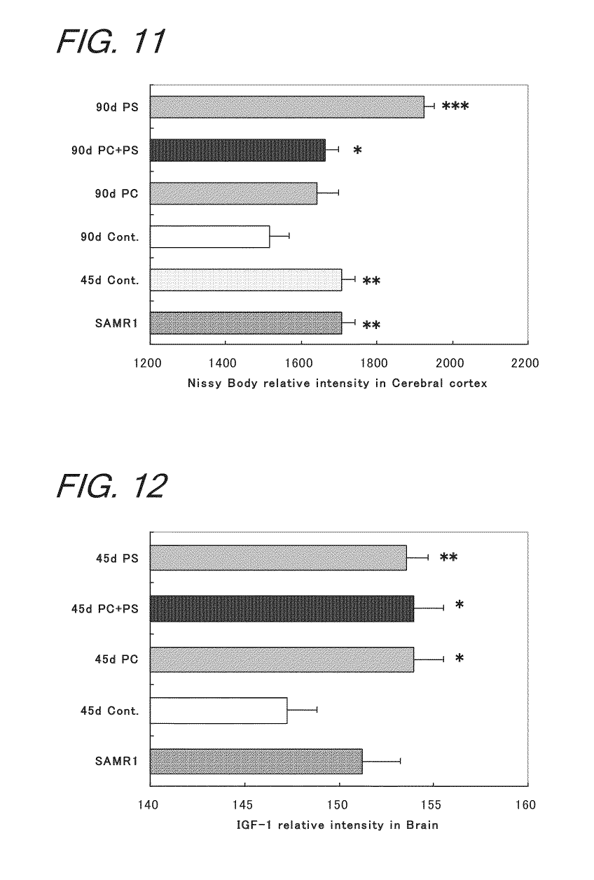

The results are shown in FIG. 12. In a comparison of the IGF-1 concentration in the brain of each 45 day group, the 45 day Cont. group demonstrated a tendency to be lower than the SAMR1 group. A significant improvement effect was demonstrated by each of the administration groups as compared to the 45 day Cont. group.

(7) Antioxidation Capability and Peroxides in the Brain

The left half brain was homogenized at low temperature in a phosphoric acid buffered physiological saline solution (PBS) and then dissolved in a buffer solution [20 mM Tris (pH 7.5), 150 mM NaCl, 1% Triton X-100], the solution was centrifuged for 20 minutes at 12,000 rpm, and then the SOD activity and MDA concentration in the brain was measured using the supernatant. SOD (Superoxide Dismutase) activity was measured using a Total Superoxide Dismutase Assay Kit with WST-1 (Beyotime Institute of Biotechnology, China). The MDA (Malondialdehyde) concentration was measured using Lipid Peroxidation MDA Assay Kit (Beyotime Institute of Biotechnology, China).

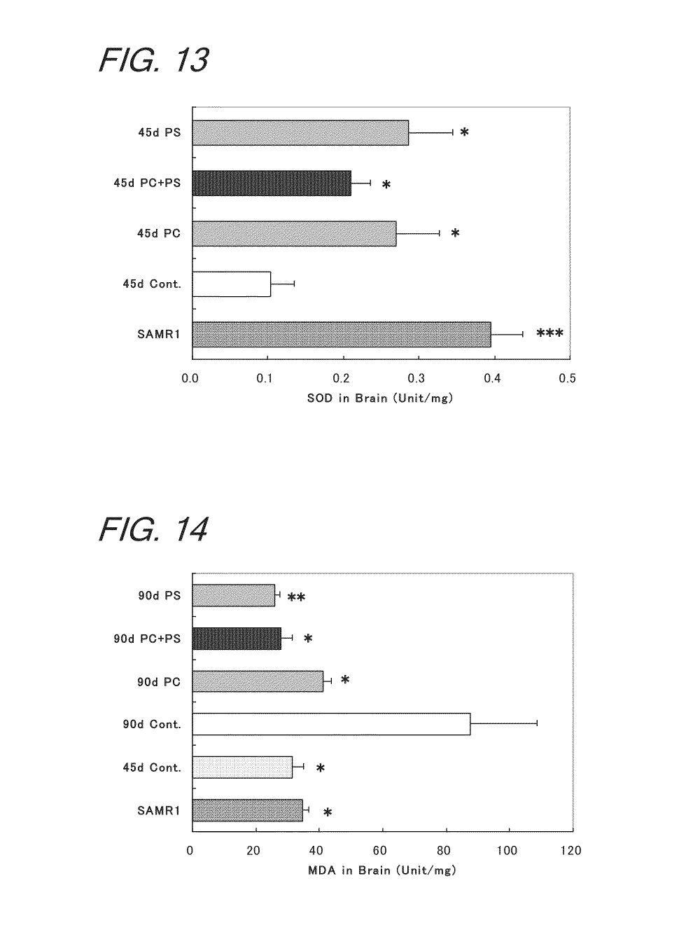

A graph of the SOD concentration in the brain for each group is shown in FIG. 13. When the SOD concentration in the brain of each 5 day group was compared, the 45 day Cont. group was much lower compared to the SAMR1 group, and all of the administration groups demonstrated significant improvement effects as compared to the 45 day Cont. group.

A graph of the MDA concentration in the brain for each group is shown in FIG. 14. When comparing the concentration of MDA in the brain for each 90 day group, the MDA concentration of the 90 day Cont. group was much higher than the SAMR1 group and the 45 day Cont. group. A significant improvement effect was demonstrated by each of the administration groups as compared to the 90 day Cont. group.

Note, in the graphs showing the results of the test example 1, the significant difference is shown by the following indications: *) p<0.05, **) p<0.01, ***) p<0.001 (Mean±SE, T test, vs Control).

### Test Example 2

Brain Atrophy Preventing Test

A brain atrophy preventing and brain function improving test of phospholipids was performed using Senescence-accelerated mouse P10 (SAMP10). The effect of a composition containing phosphatidylserine was evaluated.

(1) Test Animal

In the past, SPF animals (specific pathogen free animals, SPF) were used. SAMP10 (2 month old, male) animals for the control group and each administration group and SAMR1 (for accurate comparison group, 2 month old, male) were procured and raised under the same conditions as test example 1.

After acclimatizing for 7 days, each animal was randomly grouped as shown below.

SAMR1 group: 10 to 11 animals/groups.

SAMP10 group: 13 animals/group (after testing, the number of surviving animals in each group was: 11 to 13 animals).

(2) Testing Process

The following substances were used as the administration samples:

Phosphatidylserine derived from Krill produced in working example 3 (Krill Phosphatidylserine, K-PS: 40 wt %).

Medium Chain Triglyceride produced using palm oil as a raw material (MCT: containing 58.3% octanoic acid and 41.3% decanoic acid in fatty acid composition for the comparison group and for sample dilution).

The administration samples were provided by oral administration one time per day for 75 days, continuously. The administration groups and the administration dose were as shown below.

SAMR1 5 M group: SAMR1, before administration (five months old) SAMR1 7.5 M group: SAMR1, MCT 5 mL/kg administration (7.5 months old). SAMP10 5 M group: SAMP10, 5 months old, before administration (five months old)

SAMP10 7.5 M group: SAMP10, MCT 5 mL/kg administration (7.5 months old).

K-PS-L Group: A dose of 5 mL/kg was administered to make SAMP10 and K-PS 10 mg/kg (7.5 months old).

K-PS-H Group: A dose of 5 mL/kg was administered to make SAMP10 and K-PS 100 mg/kg (7.5 months old).

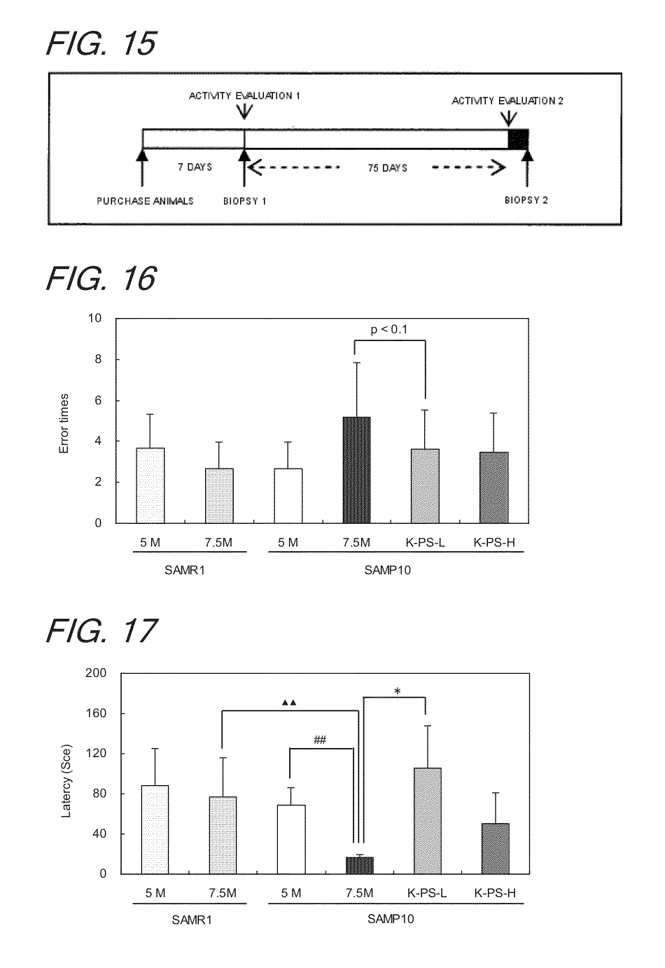

Behavior testing was performed as described below on the last day of administration, and the animal was chemically euthanized the following day, and the organs were extracted for testing. The test results are shown in FIG. 15.

(3) Step Down Test

The step down test was performed using the device illustrated in FIG. 2 using the same procedures as test example 1. The test evaluates the memory capability by recording the time until the animal first descends from the upper level to the lower level (latency time), and the number of times that the animal descends from the upper level to the lower level (error count) in 5 minutes. The test results are shown as the number of errors in FIG. 16, and the latency time in FIG. 17.

When the numbers of errors from each group are compared in FIG. 16, the K-PS-L group demonstrated significant improving effects as compared to the MCT administered 7.5 M group, and the K-PS-H group demonstrated a tendency for improvement. When the latency time from each group are compared in FIG. 17, the K-PS-L group demonstrated significant improving effects as compared to the MCT administered 7.5 M group, and the K-PS-H group demonstrated a tendency for improvement.

(4) Cerebral Neocortex

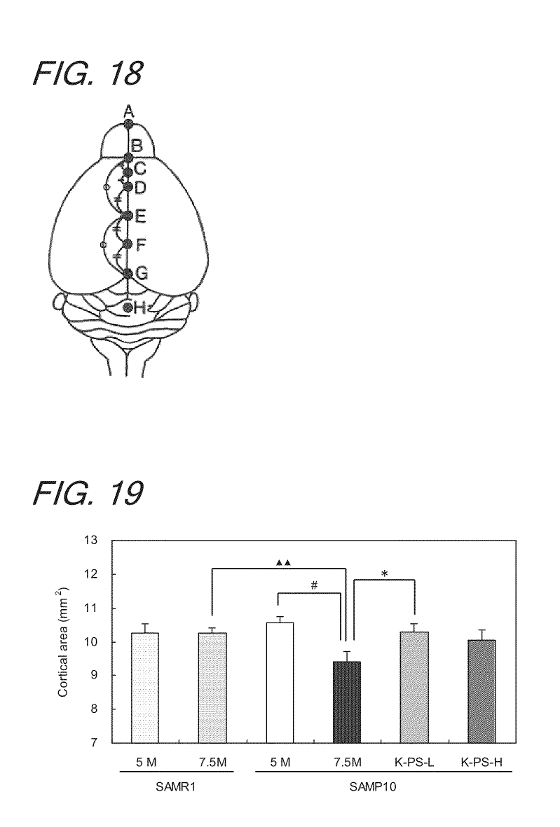

The whole brain was removed and immersed in 10% neutral buffered formalin for 2 weeks. Next, a brain tissue fragment with a thickness of 100 μm was created from tissue proximal to point C as shown in FIG. 18. The thickness of the neocortex layer and the lateral cross-sectional area of the neocortex near point C was measured using Image-Pro Plus software (version 4.0, Media Cybernetics, Bethesda, Md.).

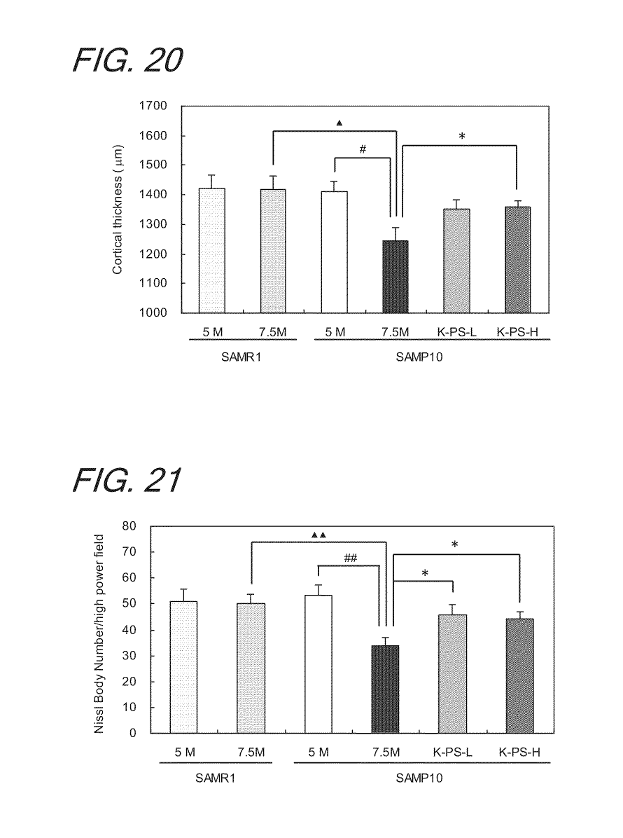

The result of the lateral cross-sectional area of the neocortex is shown in FIG. 19, and the thickness of the neocortex layer is shown in FIG. 20. In FIG. 19, the lateral cross-sectional area of the neocortex in the K-PS-L group was significantly increased as compared to the 7.5M group that was administered MCT, and a tendency for increase was observed in the K-PS-H group. In FIG. 20, the thickness of the neocortex layer in the K-PS-H group was significantly increased as compared to the 7.5 M group that was administered MCT, and a tendency for increase was observed in the K-PS-L group.

(5) Brain Nerves

The whole brain was removed and fixed using 4% para-formaldehyde solution, water was removed by a standard method, and then paraffin embedding was performed. Next, the embedded brain sample was cut to a thickness of 5 μm and dyed using hematoxylin eosine dye, and the Nissl body density of the hippocampus and the cerebral cortex was observed by fluorescence microscope using the same methods as test example 1 (5) (FIG. 21).

The embedded brain sample was cut to a thickness of 5 μm, the paraffin was removed using an alcohol solution, and then the sample was washed using phosphoric acid buffered physiological saline solution (PBS, pH 7.4) and citric acid buffered solution (10 mM sodium citrate, 0.05% Tween 20, pH 6.0), the protein bonds were broken using a 10 μg/mL protease K solution (20 mg/mL, protease K, 1 M Tris-HCl, 0.5 M EDTA, pH 8.0), and then an immunological reaction was performed overnight using Iba-1 antibodies (Anti-goat, 1:100, Abcam, United Kingdom), IGF-1 antibodies (Anti-rabbit, 1:100, Santa Cruz Biotechnology, USA) by adding 50 μL of goat blood at 4° C. Next, the immunological reaction solution was washed using a phosphoric acid buffer solution, and then dyed using 3,3′-diaminobenzidine (DAB). The intensity of a positive reaction for Iba-1 (ionized calcium binding adapter molecule 1) and IGF-1 (insulin-like growth factor 1) was measured using a fluorescence microscope.

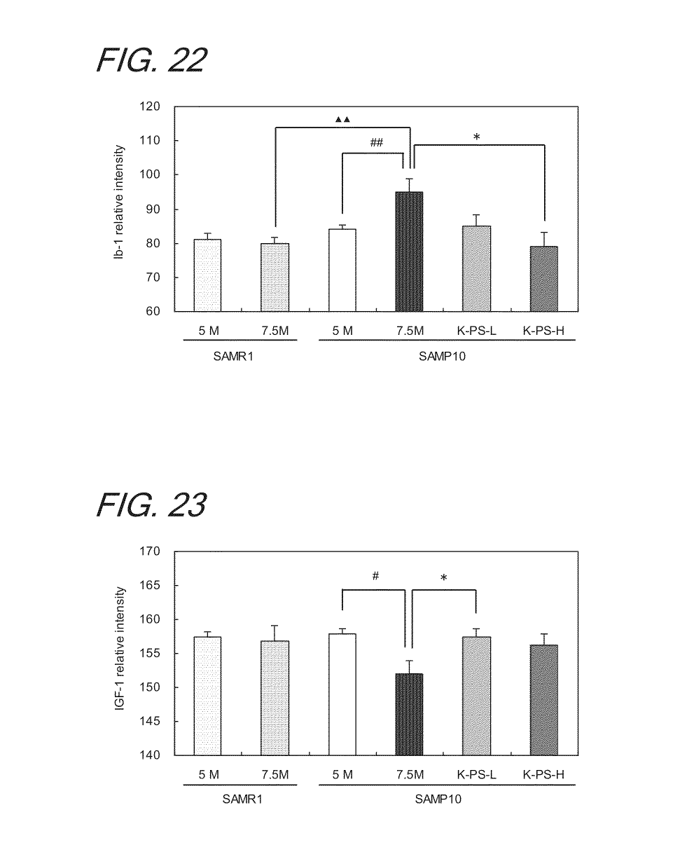

The results for the Iba-1 concentration are shown in FIG. 22, and the results of the IGF-1 concentration are shown in FIG. 23. The Iba-1 shown in FIG. 22 is a marker for microglia cells, and an increase in microglia is thought to be an indicator of aging of the brain. In the comparison mouse sample (SAMR1), there was no change in particular between five months old (5 M), and 7.5 months old (7.5 M). In the accelerated aging mouse group (SAMP10), the amount of microglia was much higher at 7.5 months old (7.5 M) as compared to 5 months old (5 M), thus indicating aging of the brain and progression of atrophy. On the other hand, in the K-PS administration groups (K-PS-L group and K-PS-H group), the increase in the amount of microglia is essentially suppressed.

The IGF-1 shown in FIG. 23 is an aging marker for the brain, and a reduction in the amount of IGF-1 is thought to indicate aging of the brain. In the comparison mouse sample (SAMR1), there was no change in particular between five months old (5 M), and 7.5 months old (7.5 M). In the accelerated aging mouse group (SAMP10), the amount of IGF-1 was much lower at 7.5 months old (7.5 M) as compared to 5 months old (5 M), thus indicating aging of the brain and progression of atrophy. On the other hand, in the K-PS administration groups (K-PS-L group and K-PS-H group), the reduction in the amount of IGF-1 is essentially suppressed.

(6) Antioxidation Capability and Peroxides in the Brain

The left half brain was homogenized at low temperature in a phosphoric acid buffered physiological saline solution (PBS) and then dissolved in a buffer solution [20 mM Tris (pH 7.5), 150 mM NaCl, 1% Triton X-100], the solution was centrifuged for 20 minutes at 12,000 rpm, and then the GSH-Px activity and MDA concentration in the brain was measured using the supernatant. GSH-Px (Glutathione peroxidase) activity was measured using GSH-Px Elisa Kit (Beyotime Institute of Biotechnology, China). The MDA (Malondialdehyde) concentration was measured using Lipid Peroxidation MDA Assay Kit (Beyotime, China).

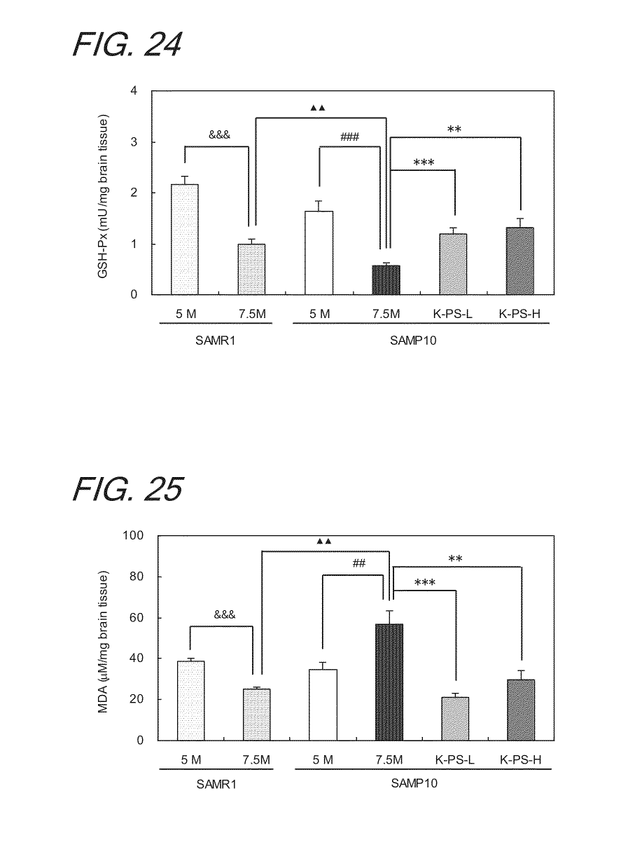

A graph of the GSH-Px concentration in the brain for each group is shown in FIG. 24, and a graph of the MDA concentration in the brain for each group is shown in FIG. 25. The GSH-Px shown in FIG. 24 is a marker that indicates antioxidation capability. In both the comparison mouse group (SAMR1) and the accelerated aging mouse group (SAMP 10), a reduction of GSH-Px was observed at 7.5 months old (7.5 M) as compared to five months old (5 M). On the other hand, in the K-PS administration groups (K-PS-L group and K-PS-H group), suppression of the reduction in the amount of GSH-Px was observed.

The MDA shown in FIG. 25 is a marker that indicates antioxidation capability. In the accelerated aging mouse group (SAMP10), the amount of MDA was much higher at 7.5 months old (7.5 M) as compared to 5 months old (5 M). On the other hand, in the K-PS administration groups (K-PS-L group and K-PS-H group) suppression of an increase in MDA or a reduction of MDA was observed as compared to five months old (5 M).

Note, in the graphs showing the results of test example 2, the significant difference is shown by the following indications: *) p<0.05, **) p<0.01, ***) p<0.001, #) p<0.05, ##) p<0.01, ###) p<0.001, ▴) p<0.05, ▴▴) p<0.01, &&&) p<0.001 (Mean±SE, T test).

What is claimed is:

1. A method for preventing brain atrophy, comprising administrating to a subject an effective amount of a phospholipid containing a highly unsaturated fatty acid as a constituent fatty acid,.

2. The method according to claim 1, wherein the highly unsaturated fatty acid is an n-3 highly unsaturated fatty acid.

3. The method according to claim 2, wherein the n-3 highly unsaturated fatty acid is eicosapentaenoic acid or docosahexaenoic acid.

4. The method according to claim 1, wherein the phospholipid is selected from the group consisting of phosphatidylcholine, phosphatidylserine, phosphatidylethanolamine, phosphatidic acid, phosphatidylglycerol, and phosphatidylinositol.

5. The method according to claim 4, wherein the phospholipid is a phosphatidylserine.

6. The method according to claim 1, wherein the phospholipid is a refined krill oil or a product of enzyme reaction with a krill oil as a substrate.

7. The method according to claim 6, wherein the refined krill oil contains 40 wt % or more of phospholipids, and a ratio of n-3 highly unsaturated fatty acid in the total fatty acid is 20 wt % or more.

8. The method according to claim 6, wherein the refined krill oil contains 90 wt % or more of diacylglycerophospholipids and 6 wt % or less of lysoacylglycerophospholipids in the phospholipid composition thereof.

9. The method according to claim 6, wherein the refined krill oil or the product of enzyme reaction with a krill oil as a substrate is:
a krill oil that is obtainable by a process comprising: obtaining a squeezed liquid by squeezing a whole krill or a part thereof, heating the squeezed liquid to a temperature at which proteins contained in the squeezed liquid coagulate, carrying out solid-liquid separation so as to separate the heated squeezed liquid into a solid component that contains lipid components and an aqueous component that contains water-soluble components, washing the resulting solid containing lipids or a dried product thereof with water, dehydrating and/or drying, and then extracting lipids from the solid containing lipids or the dried product thereof; or
a phosphatidylserine-containing oil/fat produced with the krill oil as a raw material.

10. The method according to claim 1, wherein the dosage of the phospholipid is from 1 to 10,000 mg/50 kg body weight/day.

11. The method according to claim 1, wherein the brain atrophy occurs in conjunction with aging.

12. The method according to claim 1, wherein the brain atrophy occurs in conjunction with Alzheimer's disease.
